(19) United States
(12) Patent Application Publication
(10) Pub. No.: US 2025/0057656 A1
(43) Pub. Date: Feb. 20, 2025

(54) Porous-Based Bone Replacement Materials Formed By Triply Periodic Minimal Surface Structure

(71) Applicant: Patcharapit PROMOPPATUM, Nonthaburi (TH)

(72) Inventors: Patcharapit PROMOPPATUM, Nonthaburi (TH); Atikom SOMBATMAI, Nong Bua Lamphu (TH); Saran SEEHANAM, Roi Et Province (TH); Therapong POLTUE, Khon Kaen (TH); Chatchai KARUNA, Pathum Thani Province (TH); Suppakrit KHRUEADUANGKHEM, Lampang Province (TH); Prasit PAVASANT, Bangkok (TH); Viritpon SRIMANEEPONG, Bangkok (TH); Lertrit SARINNAPHAKORN, Bangkok (TH)

(21) Appl. No.: 18/719,380

(22) PCT Filed: Dec. 15, 2022
(86) PCT No.: PCT/TH2022/000050

§ 371 Date: Jun. 13, 2024

(30) Foreign Application Priority Data
Dec. 16, 2021 (TH) 2101007868

(51) Int. Cl. A61F 2/28; A61F 2/30

(52) U.S. Cl. CPC A61F 2/28; A61F 2/30771; A61F 2002/30148; A61F 2002/30324; A61F 2002/3092

(57) ABSTRACT

Porous-based bone implants with the integration of Triply Periodic Minimal Surface, TPMS, porous architectures are designed to support the growth and proliferation of bone tissue, bone marrow, and capillaries. This disclosure intends to reduce the adverse effect of conventional implants such as bone resorption over time, which is called the “stress shielding effect”. The “stress shielding effect” is caused by the mismatch between the implant and natural bone stiffness. Triply Periodic Minimal Surface, TPMS, porous architectures exhibit interconnected pore features. The interconnection of the porous network allows the TPMS to have a higher permeability than that of other porous structures, leading to more favorable nutrient transport. In addition, many physical characteristics of the TPMS structures including surface-to-volume ratio, pore size, elastic modulus, and fluid behaviors can be controlled precisely through mathematical manipulation. As a result, TPMS-based implants could be physical features, which are vaned based on different bone regions. In other words, the medical implants may exhibit non-uniform or gradient physical features, which can match the characteristic of trabecular and cortical bones. Therefore, TPMS-based implants could adjust the features to mimic neighboring bone regions. As a result, we can achieve medical implants, which have superior mechanical and biological responses, resulting in optimal cell growth and better medical treatment.

### FIELD OF THE INVENTION

The present invention disclosure relates to the field of orthopedic implants, and more specifically to the design procedure of porous architectures, which can mimic natural bone structures. As a result, superior bone replacement structures which consider both mechanics and biomechanics aspects can be achieved.

### BACKGROUND OF THE INVENTION

According to the world population projection reported in ‘The World Population Prospects 2019’ by the Population Division of the Department of Economic and Social Affairs of the United Nations Secretariat, the world population with age over 65 is anticipated to account for 16% of the entire global population by 2050, which is the increase of 9% from 2019. Therefore, advanced medical solutions have received significant attention, which includes the integration of multidisciplinary research works from other related fields such as engineering and computer science.

In addition, thanks to the emergence of additive manufacturing (AM) or 3d-printing technology, the techniques have been adopted to manufacture various medical-related applications. The AM technologies allow the fabrication of highly complex parts, which are not buildable by conventional manufacturing technologies. For this reason, patient-specific personalized prosthetics, cutting guides, and implants can be designed and made with high precision. Furthermore, AM technology is applicable to make with various materials including polymer, ceramic, and metal. However, most medical-related devices are mostly made of metals, in which titanium alloys are used widely owing to its biocompatibility, desirable mechanical properties, and high corrosion resistance.

Nonetheless, although personalized medical devices from AM technologies are specifically designed, there still exists some drawbacks. For an instance, as the stiffness of metals is much higher than natural bone, the use of bulk metals as the implants may lead to bone resorption over time, mainly due to the stiffness mismatch. Such phenomenon is referred to as the “stress-shielding effects”. For this reason, porous architectures are gaining more interest as they can be integrated into the devices during the design process. Consequently, porous-based medical devices resemble closely the physical characteristics of natural bones.

Presently, although porous structures have been integrated with the implants, the design of porous architectures needs to consider its effect of mechanical properties and biomechanics. As a result, the implants with superior performance can be achieved. Nonetheless, the current design of porous-based implants has not considered all the mentioned criteria. For example, most porous-based implants are designed with uniform porous structures. However, the natural bone often exhibits gradient features from cortical to trabecular regions. Thus, the uniform porous may contradict the physical characteristics of natural bones.

There are two primary porous structures, which are strut-based and surface-based. Triply Periodic Minimal Surface or TPMS structure is among the surface-based type. Additionally, the physical features of both porous structures such as pore size and stiffness can be controlled by adjusting cell size, wall thickness, and relative density. In addition, the permeability and mechanical properties are among the key factors that should be considered in the design of porous implants. The permeability indicates the capability of the nutrient to transport through the porous structures. And mechanical properties are important factors for load-bearing consideration. Both permeability and mechanical properties are affected by several parameters including wall thickness, pore size, relative density, and exterior shape. However, most designs at the present often focus on a single aspect. A design criterion which determines both permeability and mechanical properties is still lacking.

In addition to the physical agreement with natural bones, the porous structures used in the current implants are uniform. In other words, pore size, wall thickness, and local density remain identical throughout the same. This characteristic is largely in contrast with the natural bones, which can be divided into dense and spongy regions. Thus, the integration of natural bone mimicry in implant designs is critically important.

For the reasons mentioned above, the inventors noticed the limitation of the metal-based implants, of which the stiffness is much greater than the natural bones since it could lead to bone resorption due to the “stress shielding effect”. Therefore, we proposed TPMS-based metal implants, in which the pore size, wall thickness, and relative density can be precisely controlled through the alteration of the mathematical equations. Consequently, the design approach would provide the metal implants with non-uniform pore size, gradient local density, and overall relative density close to that of the natural bone showing the precise transition from trabecular to cortical bone regions.

### SUMMARY OF THE INVENTION

The present invention disclosure aims to design TPMS-based porous implants. The TPMS-based porous structures are targeted to exhibit the physical characteristics, which are suitable for the growth of bone tissue, bone marrows, and blood vessels within the internal structures. The indicator for a suitable bio-environment is fluid permeability. The fluid permeability should be at least equal to that of the natural bones. As a result, the TPMS-based porous structures will exhibit better osseointegration than that of the existing commercial products.

In addition to the fluid permeability, the sizes, shapes, and numbers of pores should be comprehensively designed to meet the desired mechanical criterion. The elastic modulus of the bones and implants will be benchmarked. The compatible stiffness could prevent long-term effect such as bone resorption or implant degradation.

Lastly, the TPMS-based porous implants will be designed with the mimicry to the characteristics of the natural bones. For example, the pore size, local relative density, and overall relative density will be designed to imitate the adjacent natural bone regions. The implants, which are within the cortical bone regions, will be thicker and denser than those adjacent to the trabecular bone.

### BRIEF DESCRIPTION OF THE DRAWING

FIG. 1 Illustration of a single unit cell of TPMS, including Primitive, Gyroid, Diamond, Neovius, IWP, and FRD. The pore size, unit cell size, and wall thickness are described in (1), (2), and (3), respectively.

FIG. 2 illustration TPMS structures with constant unit cell size, relative density, pore size, and wall thickness

FIG. 3 Illustration TPMS structures with constant unit cell size while relative density, pore size, and wall thickness are varied.

FIG. 4 Illustration TPMS structures with constant relative density, while unit cell size, pore size, and wall thickness are varied.

FIG. 5 Illustration TPMS structures with non-constant unit cell size, relative density, pore size, and wall thickness

FIG. 6 Illustration TPMS structures with heterogenous structure, showing the transition from one base structure to another. The unit cell size, relative density, pore size, and wall thickness are constant throughout the sample.

FIG. 7 Illustration for the integration TPMS structures with medical devices such as dental implants

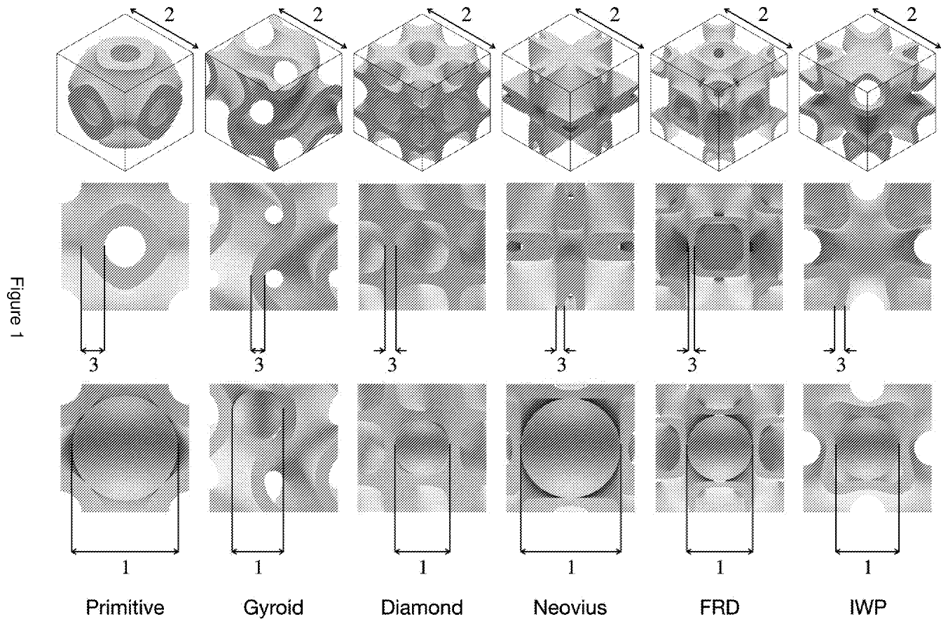

### DETAILED DESCRIPTION OF THE INVENTION

The performance of TPMS-based implants is evaluated based on their permeability and mechanical properties, in which these physical features can be precisely controlled using a mathematical equation of Triply Periodic Minimal Surface (TPMS) architectures. TPMS structures are interconnected porous architectures. The interconnectivity promotes a higher fluid transport when compared with other porous architectures. Furthermore, many physical characteristics such as surface-to-volume ratio, pore size, elastic properties, and fluid behaviors become controllable parameters. As a result, their physical characteristics can be adjusted to imitate neighboring bone regions in the human body, resulting in the implants with physical characteristic variations. As a result, local mechanical properties, permeability, and biological responses can be designed to be within the suitable ranges. To design TPMS architectures, the relative density will be varied between 0.01-1. The pore morphologies, unit cell sizes, wall thicknesses, and relative density can be designed and controlled using the following equations.

$$TPMS - Primitive \qquad ( 1 )$$

$$cos ( X ) + cos ( Y ) + cos ( Z ) = C$$

$$TPMS - Gyroid \qquad ( 2 )$$

$$sin ( X ) cos ( Y ) + sin ( Y ) cos ( Z ) + sin ( Z ) cos ( X ) = C$$

$$TPMS - Diamond \qquad ( 3 )$$

$$cos ( X ) cos ( Y ) cos ( Z ) - sin ( X ) sin ( Y ) sin ( Z ) = C$$

$$TPMS - Neovius \qquad ( 4 )$$

$$3 ( cos ( X ) + cos ( Y ) + cos ( Z ) ) + 4 ( cos ( X ) cos ( Y ) cos ( Z ) ) = C$$

$$TPMS - FRD \qquad ( 5 )$$

$$4 ( cos ( X ) cos ( Y ) cos ( Z ) ) - ( cos ( 2 X ) cos ( 2 Y ) + cos ( 2 Y ) cos ( 2 Z ) + cos ( 2 Z ) cos ( 2 X ) ) = C$$

$$TPMS - IWP \qquad ( 6 )$$

$$2 ( cos ( X ) cos ( Y ) + cos ( Y ) cos ( Z ) + cos ( Z ) cos ( X ) ) - ( cos ( 2 X ) + cos ( 2 Y ) + cos ( 2 Z ) ) = C$$

Where X=2πaxVL, Y=2πβyVL, Z=2πyzVL, L is unit cell size, x, y, and z are desired sample size in particular axis, α, β, and γ constants related to the unit cell size in the x, y, and z respectively. There are 5 design strategies using TPMS equation as follow.

### 1. Constant Unit Cell Size, Relative Density, Pore Size, and Wall Thickness

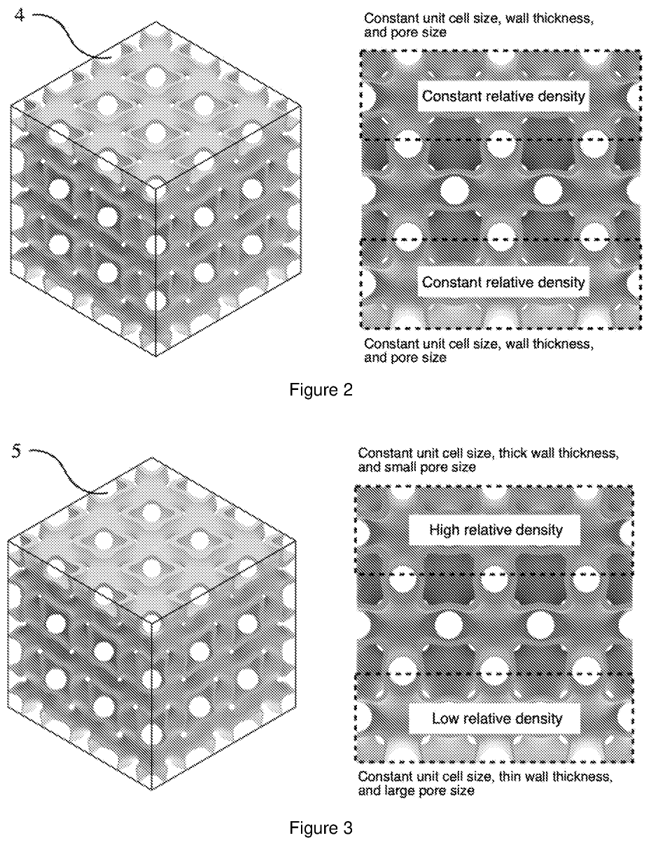

This design strategy, FIG. 2, can be achieved by selecting the base TPMS equations as shown in equations (1)-(6). Then, the unit cell size, L, is set to the desired value which results in the desirable pore size. α, β, and γ are set to the unity. In addition, two surfaces will be created at negative and positive c values. The solid TPMS can be achieved by merging surfaces that ranges in −c≤f(x,y,z)≤+c together. The c is the iso-value and is selected based on the targeted relative density. Different TPMS structures will exhibit different required c values to achieve the desired relative density.

2. Constant Unit Cell Size with Varied Relative Density, Pore Size, and Wall Thickness

This design strategy is shown in FIG. 3. To vary local relative density, the wall thickness will be varied along the z-axis. To achieve the TPMS structures with varied relative density, pore size, and wall thickness, the selected TPMS base structure from equation (1)-(6) must be solved with the constant unit cell size, L. Again, the unit cell size will be set based on the desirable pore size. And α, β, and γ are set to the unity. However, to form solid TPMS with gradient features, the merging surface will be formulated based on the non-constant level set values in the TPMS equation. The level-set values could be calculated by −(az+b)≤f(x,y,z)≤+(az+b), where a and b are constants used to specify ranges of varied local density. As the local density changes along the samples, the pore size and wall thickness are also changed. The volume enclosed within the positive and negative surfaces will form the solid TPMS. The determination of a and b will be based on the choices of different TPMS structures.

3. Constant Relative Density with Varied Unit Cell Size, Pore Size, and Wall Thickness

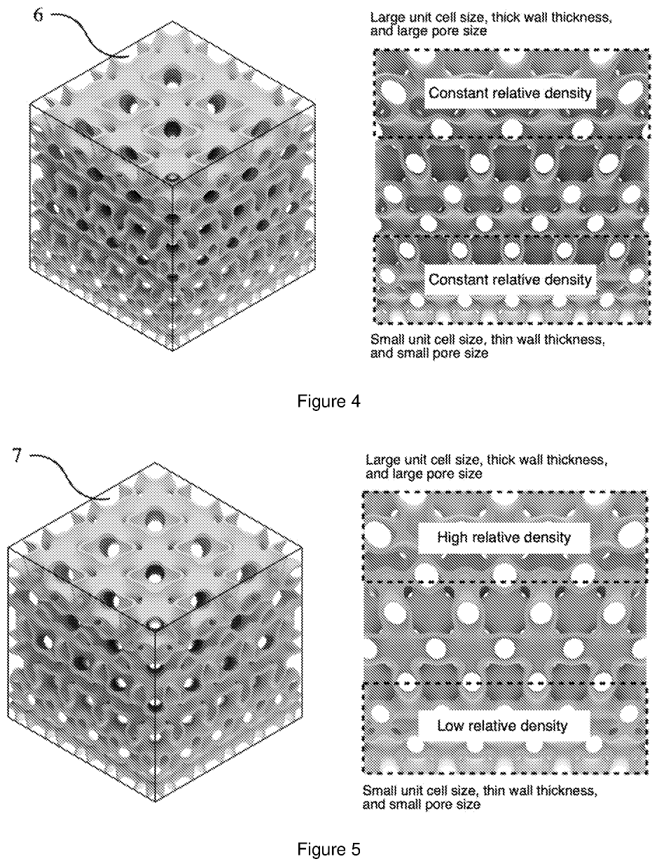

This design strategy is shown in FIG. 4. The target of this design strategy is to maintain constant local density while varying the wall thickness along the z-axis. To formulate such structures, the selected TPMS base structure from equations (1) to (6) must be solved by setting α, β, and γ as shown in equation (7)-(8).

$${α}_{( z )} = {β}_{( z )} = {k}_{1} \cdot z + {C}_{1} \qquad ( 7 )$$

$${γ}_{( z )} = \frac{{k}_{1}}{2} \cdot z + {C}_{1} + \frac{{C}_{0}}{z} \qquad ( 8 )$$

Where K_1=(m−1)/(Z_max−Z_min),C_1=−Z_min K_1+1 and C_0=½ K_1 Z_min\hat{ }2, when L_initial=mL_final. In which L_final and L_final is starting and ending unit cell size.

### 4. Non-Uniform Unit Cell Size, Relative Density, Pore Size, and Wall Thickness

This design strategy, FIG. 5, is containing non-uniform features for all physical parameters. It is achieved by applying the varied unit cell size, L, along with the previous design strategy, as fully described in (3). By changing the unit cell size, the relative density will become non-constant. The full description to control the local relative density was previously shown in (2).

5. Heterogenous TPMS Structures with Constant Unit Cell Size, Relative Density, Pore Size, and Wall Thickness

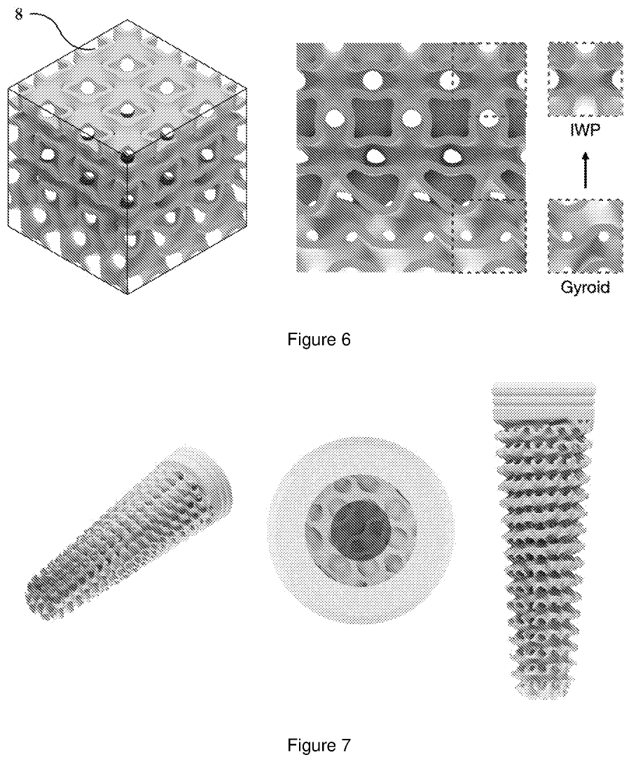

This design strategy, FIG. 6, can be fabricated by combining two TPMS base equations from (1)-(6). Followingly, the transition between two different structures will be achieved using equation (9), where ε_((x,y,z))=1/(1+e\hat{ }( [symbol] kg [symbol] _((x,y,z)))) and k are the coefficient for transitioning regime. In addition, g_(x,y,z) controls the sharpness of the transition gradient. In addition to heterogenous TPMS structure, the grading strategy from equation (9) can be combined with other grading strategies.

$${f}_{combined} = ε {f}_{surface 1 , ( x , y , z )} + ( 1 - ε ) {f}_{surface 2 , ( x , y , z )} \qquad ( 9 )$$

### THE BEST METHOD OF THE INVENTION

As referred in the detailed description of the invention.

1. Porous-based bone implants, which were designed using Triply Periodic Minimal Surface, TPMS. The TPMS structures may include Primitive, Gyroid, Diamond, Neovius, FRD, IWP, and others, in which their relative density varied from 0.1 to 1.

2. According to Claim (1), Triply Periodic Minimal Surface-based or TPMS-based bone implants have constant (i) pore size, (ii) unit cell size, (iii) wall thickness, and (iv) relative density throughout the sample.

3. According to Claim (1), Triply Periodic Minimal Surface-based or TPMS-based bone implants have (i) pore size, (iii) wall thickness, and (iv) relative density, which are varied along the sample, while (ii) unit cell size is kept constant.

4. According to Claim (1), Triply Periodic Minimal Surface-based or TPMS-based bone implants have (i) pore size, (iii) wall thickness, and (ii) unit cell size, which are varied along the sample, while (iv) relative density is kept constant.

5. According to Claim (1), Triply Periodic Minimal Surface-based or TPMS-based bone implants have (i) pore size, (ii) unit cell size, (iii) wall thickness, and (iv) relative density, which are varied along the sample.

6. According to Claim (1), Triply Periodic Minimal Surface-based or TPMS-based bone implants have the combination of two different TPMS structures in the single sample. The combination of the samples could also include the description of Claim (2)-Claim (5).
